(19) United States
(12) Patent Application Publication
(10) Pub. No.: US 2019/0038752 A1
(43) Pub. Date: Feb. 7, 2019

(54) COMPOSITION COMPRISING DUAL IONIC PH-SENSITIVE COPOLYMER FOR DELIVERING SDF-1 TOPICALLY TO THE BRAIN AND USE THEREOF

(71) Applicants: SAMSUNG LIFE PUBLIC WELFARE FOUNDATION, Seoul (KR); RESEARCH & BUSINESS FOUNDATION SUNGKYUNWAN UNIVERSITY, Gyeonggi-do (KR)

(72) Inventors: Oh Young Bang, Seoul (KR); Dong Hee Kim, Seoul (KR); Gyeong Joon Moon, Seoul (KR); Hyeon Ho Kim, Seoul (KR); Doo Sung Lee, Gyeonggi-do (KR); Young Kyu Seo, Gyeonggi-do (KR)

(21) Appl. No.: 16/057,695

(22) Filed: Aug. 7, 2018

Related U.S. Application Data
(62) Division of application No. 15/468,699, filed on Mar. 24, 2017, now abandoned.

(30) Foreign Application Priority Data
Mar. 25, 2016 (KR) 10-2016-0036108

(51) Int. Cl. A61K 47/34; C08G 18/73; C08G 18/66; C08G 18/48; C08G 18/32; A61K 9/00; A61K 38/19; A61K 9/107; B82Y 5/00

(52) U.S. Cl. CPC A61K 47/34; C08G 18/73; C08G 18/6688; C08G 18/4825; C08G 18/3293; B82Y 5/00; C08G 18/329; A61K 38/195; A61K 9/1075; A61K 9/0085; A61K 9/0019

(57) ABSTRACT

The present invention relates to a composition for topically delivering SDF-1 into the brain, in which the composition comprises a dual ionic pH-sensitive copolymer and a nerve regeneration and protective factor. In the present invention, when a dual ionic pH-sensitive copolymer containing SDF-1 as a nerve regeneration and protective factor is applied to a patient suffering from ischemic stroke as a drug carrier, it induces the effective delivery of the treatment factor to a topical lesion site, and moreover, the risk factors for adverse effects may be cancelled out, so that it can be effectively used as a novel therapeutic agent for ischemic brain diseases.

### CROSS-REFERENCE TO RELATED APPLICATIONS

This application claims the priority of Korean Patent Application No. 10-2016-0036108 filed on 03.25, 2016, in the Korean Intellectual Property Office, the disclosure of which is incorporated herein by reference.

### TECHNICAL FIELD

The present invention relates to a composition for topically delivering SDF-1 into the brain, in which the composition comprises a dual ionic pH-sensitive copolymer as a drug delivery system.

### BACKGROUND

A stroke is broadly divided into two types: an ischemic stroke, which occurs in the ischemic state of brain tissue due to an interruption of blood supply to the brain tissue, and a hemorrhagic stroke, which causes hemorrhage in the brain tissue as a blood vessel bursts. In particular, a hemorrhagic stroke is a serious disease, accounting for about 80% of all stroke patients.

Cells in the core region of cerebral ischemia caused by interruption or reduction of supply of oxygen due to interruption in blood circulation in stroke patients may begin to experience a functional disorder within a few seconds to a few minutes, and eventually will suffer irreversible damage. On the other hand, the cells around the cerebral ischemia are subject to metabolic disturbances, but they may interrupt irreversible cell damage if proper treatment is given quickly.

Numerous neuroprotectants have been tried in clinical trials for the treatment of a stroke, but have failed because of the intracerebral delivery failure and systemic adverse effects of drugs due to the blood-brain barrier. 98% of low-molecular drugs and almost all of the polymer drugs do not pass through the blood-brain barrier (Pardridge W M, Mol Interv 2003), and according to a result of investigating about more than 7,000 types of drugs through a medicine database, it has been reported that only about 5% of them act on the central nervous system (Ghose A K, Comb Chem 1999).

After the onset of an ischemic stroke, brain cell death factors such as excitotoxicity, reactive oxygen species and pro-inflammatory cytokine are expressed in the brain. On the other hand, in order to maintain the homeostasis of human body, nerve regeneration and protective factors increase, which implicitly help recovery, but these effects are limited. An effort was put to improve recovery after an ischemic stroke by artificially increasing various nerve regeneration and protective factors in the brain. However, for this, intracranial injection is necessary, and there is a limitation in clinical application due to the risk of complications.

Lesions of an ischemic stroke have various characteristics such as hypoxia, increase in reactive oxygen species (ROS), and acidosis. Among them, acidosis is the characteristic shown in the acute phase of an ischemic stroke, and its hydrogen ion concentration (pH) is topically lowered to 6 or less. Recently, in order to develop a drug delivery method using the characteristics of these lesions, studies are being made on polymers capable of converting structures dependently on the hydrogen ion concentration. Among them, the dual ionic pH-sensitive copolymer (Korean Patent Application No. 10-2013-0034710) is a synthetic polymer simultaneously including a hydrophilic and biodegradable polyethyleneglycol (PEG), a tertiary amino group cationized at an acidity of a pH of 6.8 or less, and a sulfonamido group anionized at basicity of a pH of 7.0 or more. In accordance with the change of a pH, micelles may be formed by self-assembly or collapsed. In addition, it has the characteristic that it is micellized only in neutrality, and may be demicellized at a basicity of a pH of 8.0 or more and at an acidity of a pH of 6 or less. Accordingly, this synthetic polymer may be physically bound with cationic molecules because a sulfonamido group becomes anions at basicity, and may be physically bound with anionic molecules because a tertiary amino group becomes cations at acidity. If the environment is changed from basicity to neutrality after a physical binding with cationic molecules, micelles internally containing cationic molecules may be formed. If the environment is changed to acidity, it is not only demicellized again, but also may push out cationic molecules as the characteristic of a polymer is changed to anions. Conversely, if the environment is changed from acidity to neutrality after a physical binding with anionic molecules, micelles internally containing anionic molecules may be formed. If the environment is changed to acidity, it is not only demicellized again, but also may push out anionic molecules as the characteristic of a polymer is changed to cations.

### Technical Problem

Currently, there are a variety of drugs having mechanism for treating a stroke that are currently used in clinical use, including thrombolytic agents such as tissue plasminogen activator (TPA) or urokinase, platelet inhibitors, anticoagulants, cerebral vasodilators, Ca2+ channel blockers and brain edema inhibitors (Sandercock P. et al., Br. J. Hosp. Med., 47: 731-736, 1992). However, these drugs are known to exhibit weak effects when the treatment time is delayed, fail to effectively block progress of cerebral ischemia due to acute cerebral ischemia, and exhibit side effects such as nonspecific bleeding, fibrinogen dissolution, acute reocclusion and the like (Scheinberg P. et al Stroke 25: 1290-1295, 1994).

### OBJECT OF INVENTION

In this regard, the present inventors have developed a drug carrier for delivering cationic nerve regeneration and protective factors to ischemic brain lesions using a dual ionic pH-sensitive copolymer, and confirmed that by administering it to the vein of a stroke animal model, the delivery of the cationic nerve regeneration and protective factors to lesions is improved and the nerve regeneration and protection effect actually occur, and completed the present invention.

Accordingly, an object of the present invention is to provide a composition for topically delivering SDF-1 into the brain comprising a dual ionic pH-sensitive copolymer as a drug carrier.

Another object of the present invention is to provide a composition for topically delivering SDF-1 into the brain comprising a dual ionic pH-sensitive copolymer and SDF-1.

Still another object of the present invention is to provide a composition for topically delivering SDF-1 into the brain comprising a dual ionic pH-sensitive copolymer and SDF-1 (stromal cell-derivated factor-1) encapsulated in the dual ionic pH-sensitive copolymer.

Still another object of the present invention is to provide a pharmaceutical composition for treating ischemic brain diseases comprising a composition for topically delivering the SDF-1 into the brain.

### Technical Solution

An aspect of the present invention provides a composition for topically delivering SDF-1 into the brain comprising a dual ionic pH-sensitive copolymer as a drug carrier.

Another aspect of the present invention provides a composition for topically delivering SDF-1 into the brain comprising a dual ionic pH-sensitive copolymer; and SDF-1.

Yet another aspect of the present invention provides a composition for topically delivering SDF-1 into the brain comprising a dual ionic pH-sensitive copolymer and SDF-1 (stromal cell-derivated factor-1) encapsulated in the dual ionic pH-sensitive copolymer.

Yet another aspect of the present invention provides a composition for treating ischemic brain diseases comprising a composition for topically delivering the SDF-1 into the brain.

In the present invention, the dual ionic pH-sensitive copolymer may comprise, at a main chain, a repeating unit represented by the following Chemical Formula 1, and a repeating unit represented by Chemical Formula 2 or Chemical Formula 3:

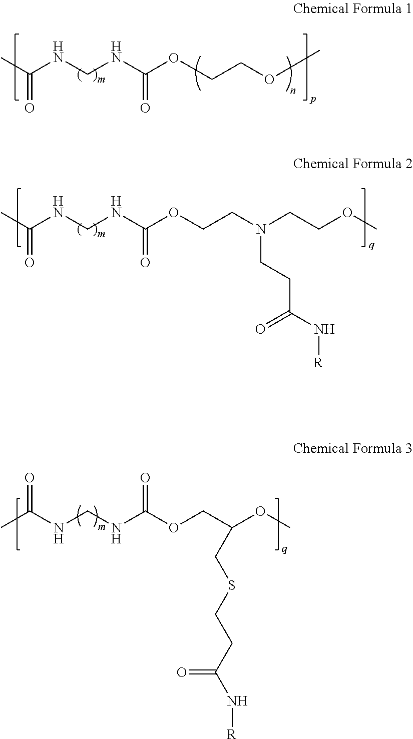

wherein m is an integer ranging from 4 to 6, n is an integer ranging from 40 to 200, p.q is from 1.1 to 1:10, preferably from 1:1 to 1:6, and each R is independently

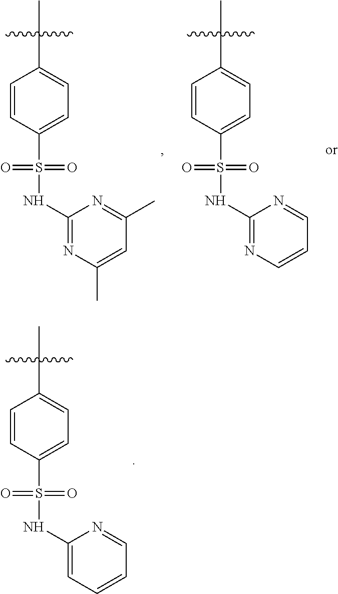

In the present invention, the dual ionic pH-sensitive copolymer may further comprise a repeating unit represented by the following Chemical Formula 4:

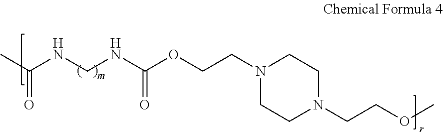

In the present invention, when the dual ionic pH-sensitive copolymer further comprises a repeating unit represented by Chemical Formula 4, p:r is 1:1 to 1:10 and m is an integer ranging from 4 to 6.

In the present invention, the number average molecular weight of the dual ionic pH-sensitive copolymer may preferably be 5,000 to 15,000.

In a composition for topically delivering SDF-1 into the brain according to the present invention, the dual ionic pH-sensitive copolymer may be present in the form of a self-assembled micelle.

Preferably, the micelle may be micellized at a pH range of from 4.5 to 8.5, and may be demicellized at a pH below 4.5 or over 8.5.

In the present invention, the ischemic brain disease may be selected from the group consisting of palsy, stroke, cerebral hemorrhage, cerebral infarction, head injury, Alzheimer's disease, vascular dementia, Creutzfeldt-Jakob disease, coma, shock brain damage, and complications therefrom.

In the present invention, the SDF-1 may be at least one selected from the group consisting of SDF-1α, SDF-1β, SDF-1γ, SDF-1δ, SDF-1ε and SDF-1φ, and preferably may be SDF-1α.

According to an exemplary embodiment of the present invention, when a composition for topically delivering SDF-1, wherein the SDF-1 is encapsulated in a dual ionic pH-sensitive copolymer, is administered to an ischemic stroke patient, it is expected that the effective delivery of the SDF-1 to the topical lesion site can be induced, thereby canceling out the risk factors due to adverse effects. Therefore, it can be effectively used as a new therapeutic agent for treating ischemic brain diseases.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 and FIG. 2 illustrate the results identifying that a dual ionic pH-sensitive copolymer may effectively deliver a target protein in an animal model of an ischemic stroke using lysozyme, which is a laboratory replacement protein bound with Cy5.5, which is a fluorescent substance;

FIG. 2 is a result of quantitative comparison of the fluorescence intensities of the ischemic ipsilateral hemisphere in FIG. 1;

FIG. 3 illustrates a process of encapsulating and micellizing SDF-1α in a dual ionic pH-sensitive copolymer;

FIG. 4 and FIG. 5 illustrate that the intracerebral delivery of SDF-1α is increased when SDF-1α is encapsulated into a dual ionic pH-sensitive copolymer and administered, rather than when only SDF-1α is administered through the tail vein. FIG. 4 shows the result of near-infrared fluorescence imaging, and FIG. 5 shows the result of quantitative analysis of FIG. 4;

FIG. 6 and FIG. 7 illustrate the results of detection of BrdU (red) and DCX (blue) through dual immunofluorescence staining method. FIG. 6 shows the result of photographing the staining region of SVZ. FIG. 7 shows the results of aggregation and quantification of the number of BrdU/DCX double positive cells of SVZ;

FIG. 8 and FIG. 9 are the results of detection of vWF through immunofluorescence staining method. FIG. 8 shows the results of fluorescence photographing of IBZ. FIG. 9 shows the result of measuring the pixels in the vWF positive region in FIG. 8;

FIG. 10 and FIG. 11 are diagrams confirming whether lysozyme encapsulated in a dual ionic pH-sensitive copolymer can be effectively delivered in an ischemic stroke animal model according to time after administration. FIG. 10 is a picture measuring the fluorescence intensity of Cy5.5 bound to lysozyme through near-infrared fluorescence imaging according to time after administration, showing that the fluorescence intensity becomes stronger if it is almost yellow. FIG. 11 is a result of quantitative comparison of the fluorescence intensities of the ischemic ipsilateral hemisphere; and

FIGS. 12 and 13 are the results of an experiment in which the delivery of lysozyme into stroke lesions was tried using Carboxymethyl Dextran (CMD), which is a substance used for drug delivery, such as a dual ionic pH-sensitive copolymer. FIG. 12 shows the results of near-infrared fluorescence imaging, and FIG. 13 shows the result of quantitative analysis of FIG. 12.

### DETAILED DESCRIPTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

One aspect of the present invention relates to a composition for topically delivering SDF-1 into the brain comprising a dual ionic pH-sensitive copolymer as a drug carrier.

Another aspect of the invention relates to a composition for topically delivering SDF-1 into the brain comprising a dual ionic pH-sensitive copolymer; and cationic nerve regeneration and protective factors.

Another aspect of the present invention relates to a composition for topically delivering SDF-1 into the brain comprising a dual ionic pH-sensitive copolymer; and SDF-1 (stromal cell-derivated factor-1) encapsulated in the dual ionic pH-sensitive copolymer.

Hereinafter, the present invention will be explained in detail.

In the present invention, the dual ionic pH-sensitive copolymer is a dual ionic pH-sensitive copolymer comprising, a tertiary amino group capable of being cationized at a low pH; and a sulfonamido group capable of being anionized at a high pH.

In the present invention, the dual ionic pH-sensitive copolymer may comprise a repeating unit represented by the following Chemical Formula 1 at its main chain; and a repeating unit represented by Chemical Formula 2 or Chemical Formula 3:

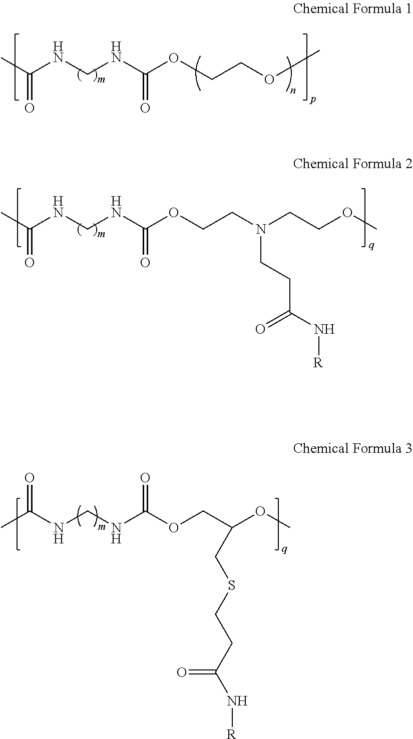

Wherein m is an integer ranging from 4 to 6, n is an integer ranging from 40 to 200, p:q is from 1:1 to 1:10, preferably from 1:1 to 1:6, and each R is independently

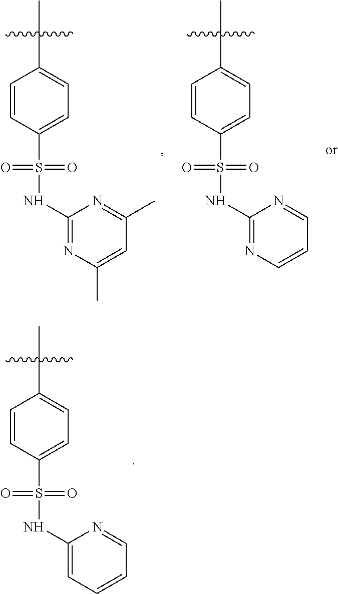

In the present invention, the copolymer is characterized in that it contains both a cationic tertiary amino group ionized at acidic of pH of 6.8 or below and an anionic sulfonamido group ionized at a basic pH of 7.0 or over. In addition, in order to improve solubility and stability, the dual ionic pH-sensitive copolymer comprises a polyethylene glycol (PEG) unit which is biodegradable compound capable of providing hydrophilicity in the repeating unit. Accordingly, the copolymer according to the present invention may form by self-assembly or collapse micelles depending on pH change. Accordingly, the copolymer of the present invention is capable of micellization/demicellization transition at two different pH depending on the pH change.

The tertiary amino group has three substituents other than hydrogen atoms bound to nitrogen, and includes a pair of non-covalent electron pairs. In general, the tertiary amino group may form a cationic salt by bonding with a hydrogen ion in a solvent using a non-covalent electron pair of a nitrogen atom. When an electron-donating group such as alkyl is substituted, its binding capacity with a hydrogen ion, that is, its basicity, is increased due to the inductive effect caused thereby. Therefore, it may be present in a cationic form in combination with a hydrogen ion under an acidic condition in which hydrogen ions may be provided.

The sulfonamido group may be converted into an anionic form under a basic condition with a weak acid having an acid dissociation constant (pKa) of about 3 to 11.

The tertiary amino and sulfonamido groups contained in the polymer of the present invention are a weak basic and a weak acidic substituents, respectively, and may be ionized in a pH-dependent manner at a region outside the pH of the living body.

In addition, in the above Chemical Formulas 1 to 3, p and q are preferably 1:1 to 1.10. More preferably, p:q may be from 1.1 to 1:6. When the ratio of p and q is outside the above range, it is difficult to control the molecular weight of the copolymer, and it is not easy to form micelles using the copolymer. In addition, when the ratio of q to p (q/p) is less than 1, the hydrophilic portion becomes large so that it is difficult to form micelles, or even if it is formed, it is dissolved in water and may be easily collapsed. On the other hand, when the ratio of q to p (q/p) exceeds 10, the balance between hydrophilicity and hydrophobicity in the molecule is lost, so that it does not exhibit dual ionic pH-sensitive micellization/demicellization or may be precipitated without forming micelles at a specific pH.

In addition, in the present invention, the dual ionic pH-sensitive copolymer may further comprise a repeating unit represented by the following Chemical Formula 4.

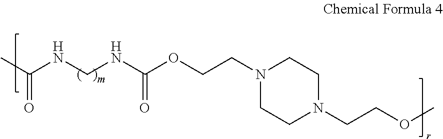

wherein m is an integer ranging from 4 to 6.

In addition, in the present invention, when the dual ionic pH-sensitive copolymer further comprises a repeating unit represented by Chemical Formula 4, it is preferable that p and r have a p:r of 1:1 to 1:10.

As described above, it is preferable to maintain the ratio of p and r within the above range in order to control the molecular weight of the copolymer and facilitate the formation of micelles. As such, when the ratio of r to p is less than 1, the hydrophilic portion becomes large so that it is difficult to form micelles, or even if it is formed, it is dissolved in water and may be easily collapsed. When it exceeds 10, the balance between hydrophilicity and hydrophobicity in the molecule is lost, so that it may be precipitated without forming micelles at a specific pH. Therefore, it is preferable to maintain the ratio of p and r within the above range.

In one preferred embodiment of the present invention, the dual ionic pH-sensitive copolymer may be poly (urethane amino sulfamethazine (PUASM) represented by the following Chemical Formula 5:

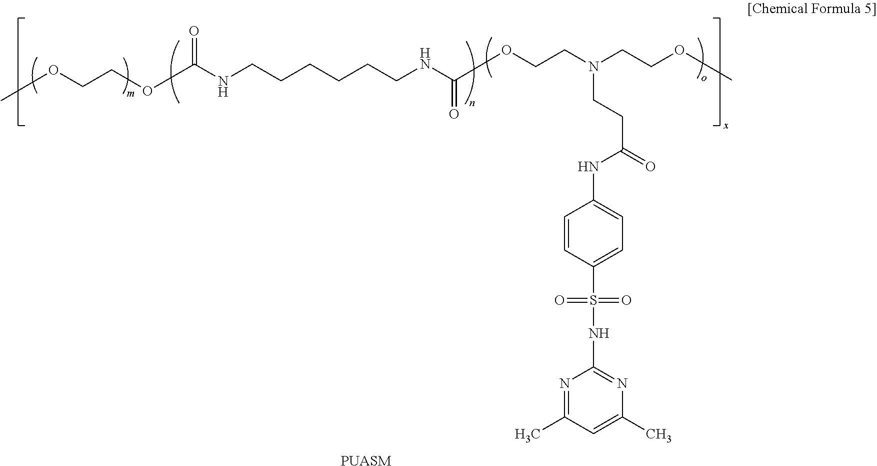

Wherein each of m, n and o is a random integer, and x depends on the molar weight of the copolymer.

The PUASM may produce micelles having a low critical micelle concentration (CMC) such as 0.0019 mg/ml by controlling pH. Considering that the micelle encapsulating drugs is diluted by blood in the blood vessel when it is injected into the body, the drug carrier exhibiting such a low CMC can be delivered to an affected area while maintaining the micelle form despite systemic administration, and so as to make a drug selectively be released depending on pH around the affected area.

In a preferred embodiment of the present invention, the dual ionic pH-sensitive copolymer may be in the form of a self-assembled micelle.

In the present invention, the dual ionic pH-sensitive copolymer is characterized in that it contains both a cationic tertiary amino group ionized at an acidic pH of 6.8 or below and an anionic sulfonamido group ionized at a basic pH of 7.0 or over. Accordingly, it may form micelles by self-assembly or collapse micelles according to pH change. Accordingly, the copolymer of the present invention is capable of micellization/demicellization transition at two different pHs depending on the pH change.

In the present invention, preferably, the micelle is micellized at a pH range of from 4.5 to 8.5, and is demicellized at pH of below 4.5 or over 8.5.

Thus, in the present invention, the pH of a solution containing the copolymer may be increased from acidic pH of 4.5 to less than 6.5 to neutral or weak basic, or is decreased from basic pH of 8.0 to greater than 8.5 to neutral or weak basic by using the dual ionic pH-sensitivity of the copolymer, and the micelles may be easily prepared. In addition, the prepared micelle may maintain a micelle form by maintaining the solution at pH from 4.5 to 8.5, preferably pH from 6.0 to 8.0.

The copolymer has a dual ionic pH-sensitivity, and is micellized or demicellized according to pH. In comparison of this with the in vivo pH, the micelle prepared according to the present invention may maintain the micelle form at a physiological pH of about 7.4 in the environment surrounding in vivo normal cells. However, under low pH conditions surrounding abnormal cells such as cancer, ischemia or inflammatory regions, the micelles may be collapsed. Accordingly, the micelles may be selectively collapsed in the lesion site at a low pH, and thus it may be used as a drug carrier by encapsulating drugs therein.

In the present invention, the diameter of the polymeric micelle is not particularly limited, but is preferably in the range of 120 to 180 nm. In addition, the polymeric micelle drug composition may be formulated in the form of an oral or a parenteral formulation, and may be prepared as intravenous, muscular, or subcutaneous injections.

In the present invention, the polymeric micelle has a diameter of 120 to 180 nm, and thus it is possible to secure a sufficient space for containing the drug therein.

The pharmaceutical composition according to the present invention may comprise nerve regeneration and protective factors, and the nerve regeneration and protective factor may preferably be SDF-1 (stromal cell-derivated factor-1).

In the present invention, the “nerve regeneration and protective factor” is a human generic recombinant protein or some peptide compositions having the above protein structure, and includes all factors which would have an effect of promoting nerve regeneration and protective effect in the brain. The nerve regeneration promoting effect means the effect of differentiation of neural stem cells and brain nerve precursor cells in the brain into proliferation, migration, nerve cells or neuroglial cells. It also includes the action that promotes neurogenesis and synaptogenesis. The nerve protective promoting effect means the effect capable of suppressing brain cell death caused by an ischemic stroke and minimizing brain damage.

In the present invention, the SDF-1 (stromal cell-derivated factor-1) chemokine (chemotactic cytokine) is involved in both basal trafficking and inflammatory reactions, and consists primarily of a superfamily of small (8-10 kDa) cytokines that activate seven transmembranes and G-protein-coupled receptors that function as leukocyte chemoattractants and activators.

Stromal cell-derived factor-1α, SDF-1α and its two allotropes (β, γ) are small chemotactic cytokines belonging to an intercrine family. Its members activate leukocytes and are often induced by pro-inflammatory stimuli such as lipopolysaccharide, TNF, or IL-1. These intercrines are characterized by the presence of four preserved cysteines, which form two disulfide bonds. They may be classified into two subfamilies.

In CC subfamily including beta chemokine, these cysteine residues are adjacent to each other. In CXC subfamily including alpha chemokine, they are independent by intervening amino acids. The SDF-1 protein belongs to the latter group. SDF-1 is a natural ligand of the CXCR4 (LESTR/fusin) chemokine receptor. These alpha, beta, gamma allotropes are the result of alternative cutting and binding of a single gene. The alpha form is derived from exons 1-3, whereas the beta form retains additional sequences from exon 4. The first three exons of SDF-1γ conform to the exons corresponding to SDF-1α and SDF-1β. The fourth exon of SDF-1γ is located 3200 bp downstream from the third exon on the SDF-1 locus, and is located between the third and fourth exons of SDF-1β.

Three new SDF-1 allotropes, SDF-1 delta, SDF-1 epsilon and SDF-1 pi have recently been reported (Yu et al., 2006). The SDF-16 allotrope is alternatively cut and bound at the final codon of an SDF-1α open reading frame, and yields 731 base pair introns, wherein the terminal exon of SDF-1α is split into two parts. The first three exons of SDF-1ε and SDF-1φ are 100% identical to the corresponding exons of SDF-1β and SDF-1γ allotropes.

The SDF-1 gene is expressed ubiquitously, except for in blood cells. It acts on lymphocytes and monocytes in vitro, but does not act on neutrophils, and is a very potent chemoattractant for mononuclear cells in vivo. In vitro and in vivo, SDF also functions as a chemoattractant for human hematopoietic progenitor cells expressing CD34.

In the present specification, “SDF-1” may be SDF-1 activity, for example, full-length matured human SDF-1α or fragments thereof having binding activity to the CXCR4 receptor. In the present specification, “SDF-1” may be an optional SDF-1 derived from an animal, for example, a murine, a bovine, or a rat SDF-1 if the identity sufficient to maintain SDF-1 activity exits.

In the present specification, “SDF-1” may also be a biologically active mutein and a fragment of SDF-1, such as a naturally occurring allotrope (isoform). Six alternatively cut and bound transcriptome mutants of the gene encoding the different allotropes of SDF-1 have been reported (SDF-1 allotropes α, β, γ, δ, ε, φ).

In the present specification, “SDF-1” also encompasses its allotropes, muteins, fusion proteins, functional derivatives, active fractions, fragments or salts. These allotropes, muteins, fusion proteins or functional derivatives, active fractions or fragments retain the biological activity of SDF-1. Suitably, they possess improved biological activity as compared to wild-type SDF-1.

In particular, “SDF-1” retains human matured allotrope SDF-1α, human mature SDF-1β, human mature SDF-1γ, human mature SDF-1-δ, human mature SDF-1ε, human mature SDF-1φ; additional N-terminal methionine, and encompasses human matured allotrope SDF-1α; fragments of SDF-1α, for example, the form of amino acid residues 4-68 of human mature SDF-1α, amino acid residues 3-68 of human mature SDF-1α, amino acid residues 3-68 of human mature SDF-1α having additional N-terminal methionine. In addition, SDF-1 may be a fusion protein including SDF-1 polypeptide as defined previously operably connected to at least one amino acid sequences selected from heterologous domains, for example, extracellular domains of membrane-bound proteins, immunoglobulin invariant regions (Fc region), multimerization domain, export signals, and tag sequences (such as a tag that assists purification by affinity; HA tag, histidine tag, GST, FLAAG peptide, or MBP).

In a preferred embodiment of the present invention, SDF-1 is SDF-1α.

The pharmaceutical composition for treating ischemic brain diseases of the present invention may encapsulate drugs other than SDF-1 in the micelle. The drug may be used without particular limitation, and the nonlimited examples thereof include anticancer drugs such as paclitaxol, doxorubicin, docetaxel, chlororambucyl, insulin, exendin-4, protein drugs such as human growth hormone (hGH), erythropoietin (EPO), granulocyte colony stimulating factor (G-CSF), granulocytemacrophage stimulating factor (GM-CSF) and bovine serum albumin (BSA), gene medicine such as DNA, antibacterial agents, steroids, anti-inflammatory analgesic agents, sex hormones, immunosuppressants, antiviral agents, anesthetics, antiemetic drugs, antihistamines, etc., and may be drugs such as antibacterial agents, steroids, anti-inflammatory analgesic agents, sex hormones, immunodepressants, antiviral agents, anesthetics, antiemetic drugs or antihistamines, etc., and ordinary additives known in the pertinent art in addition to the above-discussed ingredients.

In the present invention, there is an advantage in that the copolymer may be cationized or anionized depending on pH, and is capable of encapsulating both cationic and anionic drugs because it can make micellization/demicellization transition in acidity and basicity. For example, if the drug to be encapsulated is anionic, the drug and copolymer are mixed under acidic conditions to induce ionic interaction between the anionic drug and the cationized copolymer, and thus micelles encapsulating an anionic drug may be prepared by increasing pH and allowing the copolymer to form micelles. Conversely, when the drug to be encapsulated is a cation, the drug and copolymer are mixed under basic conditions to induce ionic interaction between the cationic drug and the anionized copolymer, and thus micelles encapsulating a cationic drug may be prepared by decreasing pH and allowing the copolymer to form micelles.

Meanwhile, molecular image markers or contrast agents which may be encapsulated inside the polymeric micelles through ionic interaction are also included in the category of the drug, and the drug carrier encapsulating the molecular image marker or the contrast agent may be used for diagnosis of diseases. Non-limiting examples of the molecular image markers or contrast agents include pyrene, RITC, FITC, ICG (indocyanine green), iron oxide, manganese oxide, and the like. In addition, the molecular imaging marker or contrast agent may be encapsulated inside the micelle together with the above-mentioned drug or by being labeled on the above-mentioned drug, which has the advantage of simultaneously diagnosing and treating the disease.

In addition, the copolymer of the present invention may be prepared by a method comprising the following steps:

mixing i) a compound represented by the following Chemical Formula 6, ii) a compound represented by the following Chemical Formula 7, iii) a compound represented by the following Chemical Formula 8 or 9, and iv) alternatively, a compound represented by the following Chemical Formula 10 with anhydrous solvent; and adding a catalyst to perform a urethane bond forming reaction:

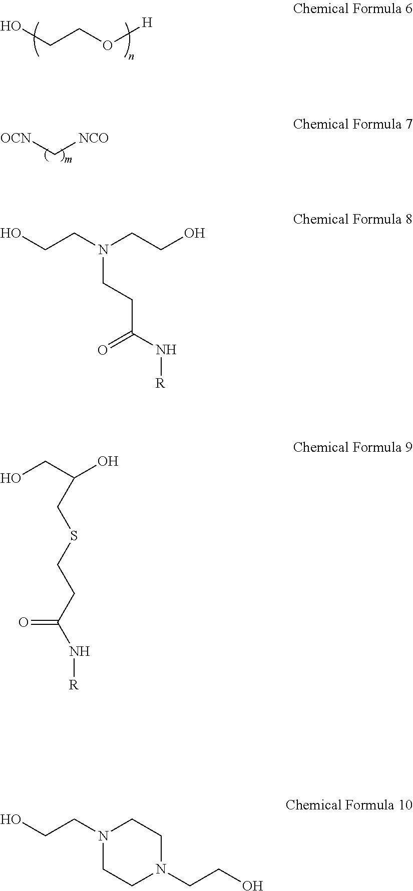

wherein m is an integer ranging from 4 to 6, n is an integer ranging from 40 to 120, and each R is independently

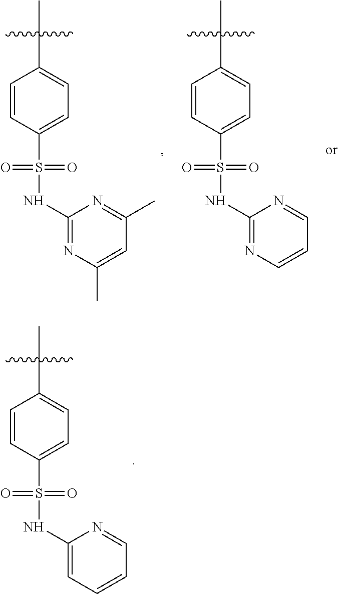

The copolymer of the present invention may be prepared by reacting a monomer containing the reactants, which are a hydroxy group or isocyanate group, at both terminals as a reactor to form a urethane bond between the hydroxyl group and the isocyanate group by a catalytic reaction. Among the compounds used as reactants in the present invention, the compounds represented by the above Chemical Formulas 5 and 7 to 9 comprise a hydroxyl group as a reactor at both terminals, and the compound represented by Chemical Formula 6 comprises an isocyanate group as a reactor at both terminals. Accordingly, a copolymer may be prepared by adding a suitable catalyst and controlling the reaction conditions to induce urethane bond formation after mixing the above compounds in an appropriate solvent. At this time, since only the compound represented by Chemical Formula 6 contains the isocyanate group as a reactor, the prepared copolymer may form a random copolymer in which the compounds represented by Chemical Formulas 5 and 7 to 9 are randomly connected by the media of the compound represented by Chemical Formula 6.

In the present invention, the method for preparing the copolymer may use the urethane bond forming reaction known in the pertinent art without limitation.

Preferably, the anhydrous solvent used in the method for preparing the copolymer of the present invention may be anhydrous dimethylformamide (DMF), methyl ethyl ketone (MEK) or dimethylsulfoxide (DMSO), but is not limited thereto. In addition, any solvent which may be used in the preparation of polyurethane may be used without limitation.

Preferably the catalyst may be dibutyltin diaurate, dioctyltin oxide, bismuth octanoate or 1,4-diazabicyclo[2.2.2]octane(1,4-diazabicyclo[2.2.2]octane).

According to one specific embodiment of the present invention, the lysozyme and SDF-1a having a positive net charge at physiologically active pH and the dual ionic pH-sensitive copolymer are respectively dissolved in PBS, wherein they are mixed and stirred after adjusting the pH to 9.0, and pH was lowered to 7.4 to form micelles. Thus, the lysozyme and SDF-1a prepare micelles encapsulated therein.

The ischemic brain disease is preferably at least one selected from the group consisting of palsy, stroke, cerebral hemorrhage, cerebral infarction, head injury, Alzheimer's disease, vascular dementia, Creutzfeldt-Jakob disease, coma, shock brain damage, and complications thereof, more preferably stroke or cerebral infarction.

As discussed above, a pharmaceutical composition comprising a dual ionic pH-sensitive copolymer, and a stromal cell-derived factor-1 (SDF-1) encapsulated in the copolymer effectively delivers SDF-1 to a topical lesion site of ischemic brain tissue, and by selectively releasing SDF-1, which is the nerve regeneration and the protective factor, at the lesion where pH has been topically lowered, the effective delivery of drugs may be induced, and moreover, the risk factors for adverse effects may be cancelled out.

The pharmaceutical composition of the present invention may be administered in various formulations of oral and parenteral administration in actual clinical administration. In case of preparations, it may be prepared using diluents or excipients such as fillers, extenders, binders, wetting agents, disintegrating agents and surfactants which are commonly used.

In particular, the pharmaceutical composition of the present invention is preferably an injectable preparation or an oral preparation.

Preparations for parenteral administration include sterilized aqueous solutions, non-aqueous solvent, suspensions, emulsions, lyophilized preparations and suppositories. As non-aqueous solvent and the suspension solvent, propyleneglycol, polyethyleneglycol, vegetable oil such as olive oil, injectable ester such as ethyl oleate, and the like may be used. As a base for suppositories, witepsol, macrogol, tween 61, cacao butter, laurinum, glycerol, gelatin and the like may be used. The pharmaceutical composition of the present invention may be administered by subcutaneous injection, intravenous injection, intraperitoneal administration, or intramuscular injection at the time of parenteral administration.

The pharmaceutical composition of the present invention may be prepared by adding a pharmaceutically acceptable carrier. For the contents of preparations, the documents of Remington's Pharmaceutical Science (latest edition), Mack Publishing Company, Easton Pa. may be a reference.

The pharmaceutically acceptable carrier means those usually used in the preparation of a pharmaceutical composition for a person skilled in the art to which the medical invention pertains. For example, it includes lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinyl pyrrolidone, water, methylhydroxybenzoate, propylhydroxybenzoate, talc, magnesium stearate, and mineral oil. In addition, the pharmaceutically acceptable carrier also includes diluents or excipients such as fillers, extenders, binders, wetting agents, disintegrating agents, surfactants, and the like. However, the present invention is not limited to the above listed pharmaceutically acceptable carriers and the like, and these are merely examples.

The dosage of the cationic nerve regeneration and the protective factor contained in the pharmaceutical composition may vary depending on the condition and body weight of a patient, the degree of disease, the form of drug, the administration route and the period, but may be appropriately selected depending on the cases. In the case of administration, it may be applied once a day or may be divided into several times.

The pharmaceutical composition may be applied to mammals such as humans in various routes, for example, by oral, intravenous, intramuscular, or subcutaneous injection.

In addition, the pharmaceutical composition of the present invention may contain at least one type of active ingredient showing the effect of preventing or treating an ischemic brain disease in addition to the SDF-1.

In addition, the pharmaceutical composition of the present invention may be used alone, or in combination with methods using surgery, hormone therapy, drug therapy and biological response modifiers, for the improvement, alleviation, treatment or prevention of an ischemic brain disease.

Hereinafter, the constitutions and effects of the present invention will be described in more detail through examples. These embodiments are only for illustrating the present invention, and the scope of the present invention is not limited by these embodiments.

### Preparation Example 1: Preparation of Dihydroxyl Aminosulfamethazine (DHASM) Monomer

Step 1) Synthesis of Sulfamethazine Acrylate (SMA)

Sulfamethazine (SM) and sodium hydroxide were added to a 250 ml one-neck round-bottom flask at an equivalence ratio of 1:1.1, and a 1:1 mixed solvent of deionized water and acetone is dissolved to make a concentration of 10%. The reaction flask was immersed in an ice-bath and cooled to 0□. 1.1 equivalents of acryloyl chloride (AC) was added drop by drop with stirring the solution. At this time, the reaction temperature was maintained at 0□ for 2 hours, and then the temperature was raised to room temperature and further maintained for 1 hour. The product was filtered and washed several times with a sufficient amount of deionized water. Thereafter, it is dried in a vacuum oven for 48 hours to obtain SM-A.

Step 2) Synthesis of Dihydroxyl Amino Sulfamethazine Monomer

SM-A synthesized in Step 1 and diethanolamine (DEA) were mixed in a 250 ml one-neck round-bottom flask at a molar ratio of 1:1. Anhydrous N, N′-dimethylformamide (DMF) was added to the flask so that the concentration of the reactant would become 10% by weight to dissolve the reactant. The flask was reacted in an oil-bath at 50□ with constant stirring for 12 hours and the solvent was concentrated by vacuum evaporation and precipitated in excess diethyl ether. The precipitation was repeated two times and the prepared dihydroxyl amino sulfamethazine monomer was filtered and dried in a vacuum for 48 hours prior to use.

### Preparation Example 2: Synthesis of a Dual Ionic pH-Sensitive Poly(Urethane Aminosulfamethazine) (PUASM) Random Copolymer

DHASM monomer synthesized in the Preparation Example 1 and 1,4-bis(2-hydroxyethyl)piperazine (HEP) were mixed with polyethylene glycol in a 250 ml two-neck round-bottom flask, and dried under vacuum using dry nitrogen, and then 90 ml of anhydrous DMF was added. After dissolving the reactant, DBTL dissolved in anhydrous CHCl3 of the same volume as hexamethylene diisocyanate (HDI) or tetramethylene diisocyanate (TDI) was added and the reaction was additionally continued for 3 hours. Finally, the reaction solution was concentrated by vacuum evaporation and precipitated in excess diethyl ether. The precipitated product was filtered and dried in a vacuum for 48 hours. The reactants were used in combination with various molar ratios as shown in Table 1 below.

TABLE 1

| Specimen name | PEG | HDI | DHASM | HEP | TDI |
|---|---|---|---|---|---|
| PUASM 6 (32) | 1 | 6 | 3 | 2 | — |
| PUASM 6 | 1 | 6 | 4 | 1 | — |
| PUASM 6 | 1 | 6 | 5 | — | — |
| TDI-PUASM 6 (32) | 1 | — | 3 | 2 | 6 |
| TDI-PUASM 6 (41) | 1 | — | 4 | 1 | 6 |
| TDI-PUASM 6 | 1 | — | 5 | — | 6 |
| PUASM 11 (28) | 1 | 11 | 2 | 8 | — |
| PUASM 11 (37) | 1 | 11 | 3 | 7 | — |
| PUASM 11 (46) | 1 | 11 | 4 | 6 | — |
| PUASM 11 (55) | 1 | 11 | 5 | 5 | — |
| PUASM 11 (64) | 1 | 11 | 6 | 4 | — |

‘PEG’ in the Table 1 is polyethylene glycol, ‘HDI’ is hexamethylene diisocyanate, ‘DIHASM’ is dihydroxyl amino sulfamethazine, ‘HEP’ is 1,4-bis(2-hydroxyethyl)piperazine, and TDI is tetramethylene diisocyanate.

In particular, PUASM 6 was used in one embodiment of the present invention, and the molar ratios of the compositions are PEG:1, HDI:6, and DHASM:5.

### Example 1: Production of an Ischemic Stroke Animal Model

In order to confirm the possibility of the pharmaceutical composition according to the present invention, an animal model of an ischemic stroke was first created through surgical operation. In this animal model, SD rats weighing 280 g to 310 g were anesthesia-induced and cervical incised, and then nylon suture coated with non-toxic silicone is inserted into internal carotid artery through the right carotid artery to induce ischemic stroke by blocking the right middle cerebral artery.

### Example 2: Determination of Delivery Capacity Required Amount and Dosage in SDF-1α Lesions of a Dual Ionic pH-Sensitive Polymer

An ischemic animal model was prepared as in Example 1, and after 24 hours, lysozyme to which 100 ng of Cy5.5 is bound was directly administered intracranially into the corpus striatum using a stereotactic surgery and hamilton syringe. In addition, the lysozyme to which I mg of Cy5.5 is bound was encapsulated into a dual ionic pH-sensitive copolymer, PUASM 6, and the tail vein was administered at 3 hours after the animal modeling was prepared.

In both experimental groups, they were euthanized 24 hours after the animal modeling was prepared and the brain tissue was collected, and 2 mm sections were prepared to photograph near infrared fluorescence imaging and the intensity of the Cy5.5 fluorescence wavelength was quantified.

The determination of the delivery capacity in the lesion and dosage was determined by calculating quantitative values according to the following Formula:

1 (mg)/(Y/X)=Z (mg)

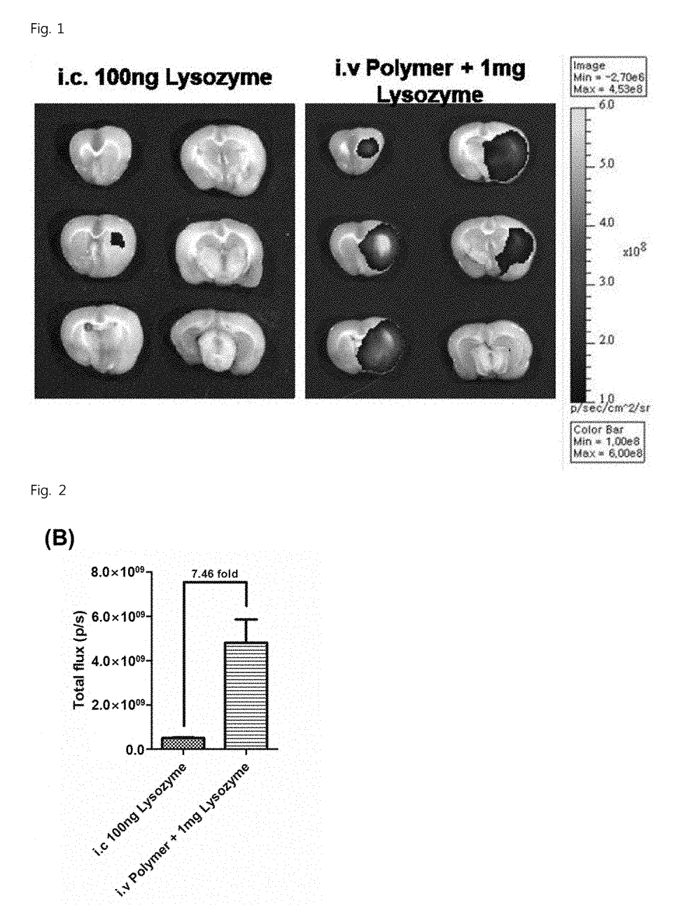

X and Y refer to fluorescence measurement values in the case of intracranial administration and fluorescence measurement values in the case of tail vein administration, respectively. Z means the tail vein dosage of lysozyme using a dual ionic pH-sensitive polymer necessary for delivering 100 ng of lysozyme into the brain. The results are shown in FIG. 1 and FIG. 2.

FIGS. 1 and 2 are drawings confirming how effectively a dual ionic pH-sensitive copolymer may deliver a target protein in an animal model of ischemic stroke using lysozyme, which is an alternative protein for experiments. In addition, this result can be utilized to determine the required dosage of SDF-1α, which is the final drug to be encapsulated in the copolymer. FIG. 1 is a picture measuring the fluorescence intensity of Cy5.5 bound to lysozyme through near-infrared fluorescence imaging, showing that the fluorescence intensity becomes stronger if it is almost yellow. FIG. 2 is a result of quantitative comparison of the fluorescence intensities of the ischemic ipsilateral hemisphere in FIG. 1.

As can be seen from FIG. 1, in the present invention, it was confirmed that the dual ionic pH-sensitive polymer effectively delivers lysozyme to the lesion site.

In addition, as can be seen from FIG. 2, the delivery of lysozyme was 7.46 times higher in the group in which lysozyme was encapsulated and treated in a dual ionic pH-sensitive copolymer as compared to the group in which lysozyme was administered solely, and it was possible to obtain the result that in order to deliver 100 ng of lysozyme into the brain, Z value of lysozyme to be encapsulated in a dual ionic pH-sensitive polymer was about 134 μg.

### Example 3: Encapsulation of Nerve Regeneration and Protective Factors in a Dual Ionic pH-Sensitive Copolymer

Stromal derived factor 1 alpha (SDF-1α) was used as a nerve regeneration and a protective factor for the present invention. Human recombinant proteins were used for the experiments. In addition, in order to facilitate the tracking to the lesion of SDF-1α, a fluorescent molecule Cy5.5 was bound and used in the experiment. Synthetic polymers were used without chemical-structurally altering the dual ionic pH-sensitive copolymers.

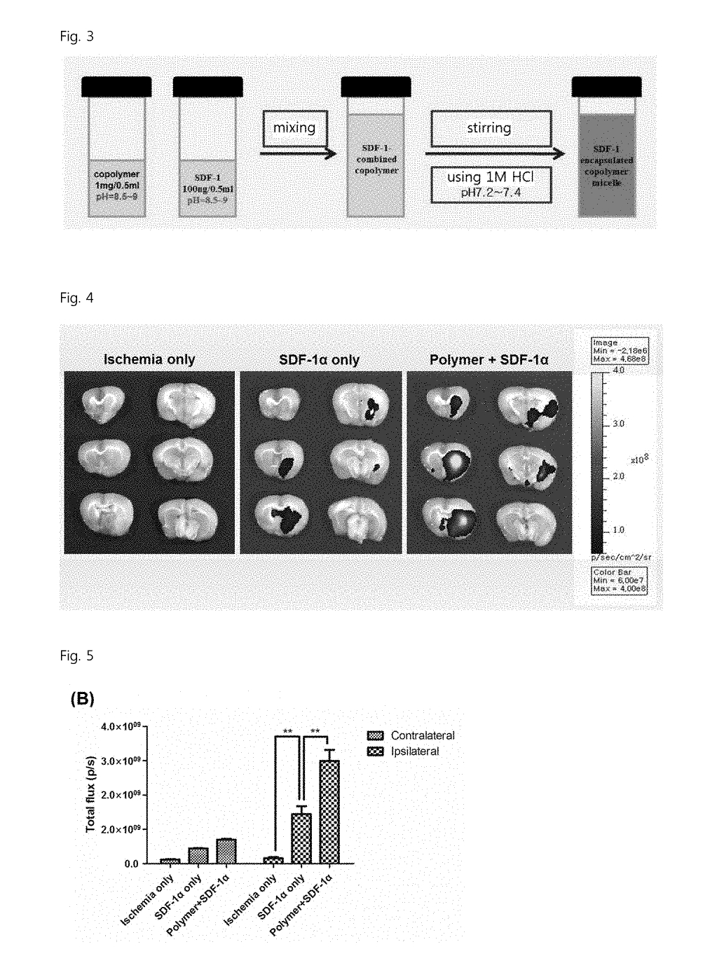

FIG. 3 shows the process of encapsulating and micellizing SDF-1α in a dual ionic pH-sensitive copolymer.

As shown in FIG. 3, mixing of the two materials is performed in phosphate buffered saline (PBS) at pH 8.5-9.0, and the pH is lowered to 7.2-7.4 using 1 M HCl to form micelles. The dosage of lysozyme was determined to be 134 μg in Example 2, but when using actual SDF-1α, an approximate value of 100 μg was determined. Among them, as to the dosage of the dual ionic pH-sensitive polymer, it has been decided to use 10 times the dosage of SDF-1α, which is the content to be encapsulated according to the prior art (Korean Patent Application No. 10-2013-0034710).

### Example 4: Delivery Effect of Nerve Regeneration and Protective Factor-Containing Copolymers to Lesions

Three hours after the production of an ischemic stroke animal model, the copolymer containing SDF-1a was administered via the tail vein of the animal. Twenty-one hours later, brain tissue was detected and 2 mm sections were prepared and quantitatively compared the strength of a fluorescence wavelength of Cy5.5 via near-infrared fluorescence imaging. As a comparative group, only Cy5.5-bound SDF-1a was dissolved in PBS and administered by the same method. In the control group, only PBS was administered. The results are shown in FIG. 4 and FIG. 5.

FIGS. 4 and 5 are drawings showing the results of increasing the intracerebral delivery of SDF-1α when SDF-1α is encapsulated and administered in a dual ionic pH-sensitive copolymer rather than when only SDF-1a is administered via the tail vein. FIG. 4 shows the result of near-infrared fluorescence imaging, and FIG. 5 shows the result of quantitative analysis of FIG. 4.

As shown in FIG. 4 and FIG. 5, it may be confirmed that when the SDF-1α was encapsulated and administered to the dual ionic pH-sensitive copolymer, the amount of SDF-1α delivered to the lesion was increased by two-fold as compared to when only SDF-1α was administered.

### Example 5: Therapy Effect of a Copolymer Containing a Nerve Regeneration and a Protective Factor

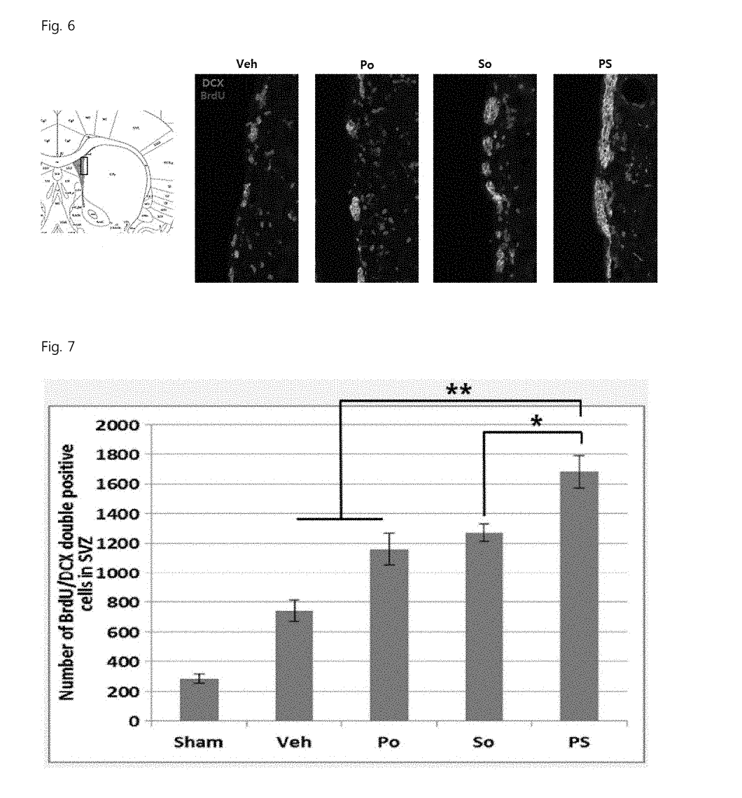

In the present invention, in order to confirm the nerve regeneration and protective effect, the copolymer encapsulating SDF-1α was administered via the tail vein for 3 hours after the preparation of the ischemic stroke animal model, and the euthanasia was induced a week later and the specimen was collected. However, unlike Example 3, SDF-1α to which Cy5.5 is not bound was used bromodeoxy uridine (BrdU) was administered every day for the period from administration to euthanasia in order to confirm the effect of nerve regeneration. As a control group for the experiment, a normal group (Sham) that does not induce stroke, a group that solvent PBS is administered, a group that only a copolymer is administered and a group that only SDF-1α is administered were additionally performed Nerve regeneration and protective effects were confirmed by immunofluorescence staining method of brain tissue sections. Nerve regeneration effect detects the proliferation of neural progenitor cells by using antibodies against BrdU and doublecortin. The neovascularization effect detects the increase in capillaries by using an antibody against von Willebrand factor (vWF) The results are shown in FIGS. 6 to 9.

FIGS. 6 and 7 show the results of detection of BrdU (red) and DCX (blue) through dual immunofluorescence staining method. FIG. 6 shows the result of photographing the staining region of SVZ. FIG. 7 shows the results of aggregation and quantification of the number of BrdU/DCX double positive cells of SVZ.

As shown in FIGS. 6 and 7, delivered SDF-1α stimulates the proliferation of neural stem cells present in the subventricular zone (SVZ), and induces differentiation into neural progenitor cells.

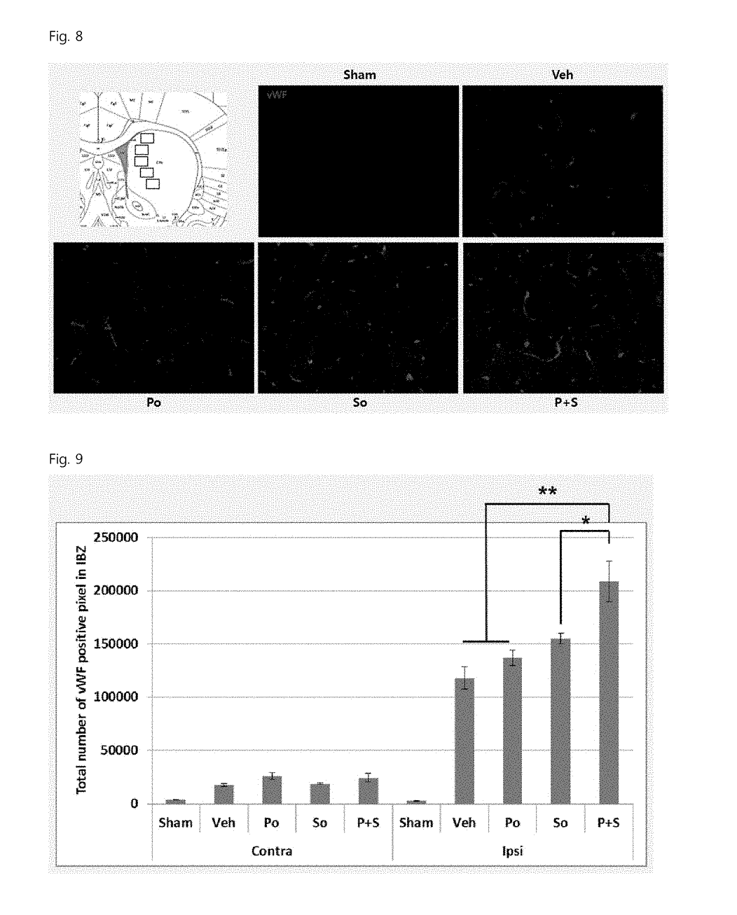

FIGS. 8 and 9 are the results of detecting vWF through immunofluorescence staining method. FIG. 8 shows the results of fluorescence imaging of IBZ. FIG. 9 shows the result of measuring the pixels in the vWF positive zone in FIG. 8.

As shown in FIGS. 8 and 9, it was confirmed that the increase in neovascularization of an ischemic border zone (IBZ) was induced.

### Example 6: Capacity Evaluation of Drug Delivery Ability in Ischemic Stroke of Dual Ionic pH-Sensitive Copolymer Using Lysozyme

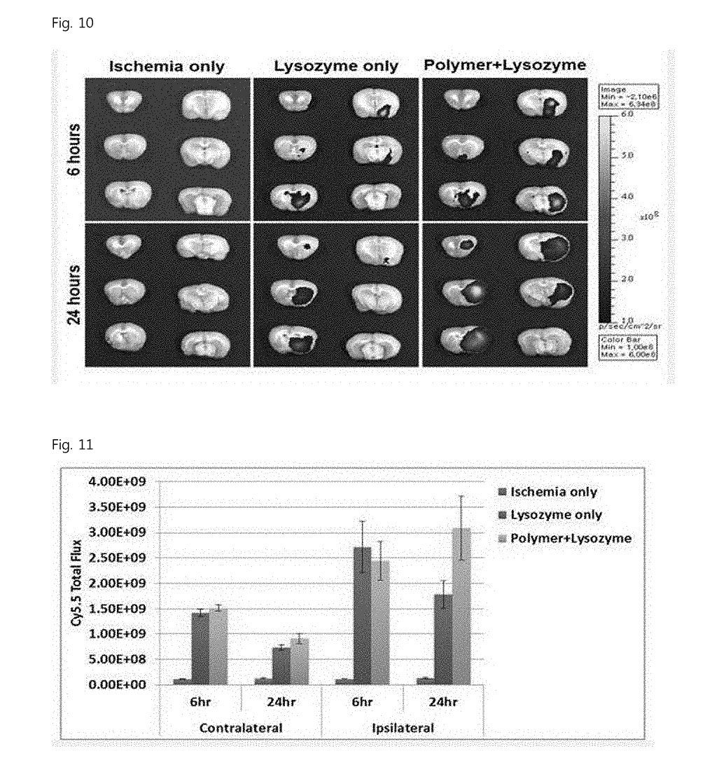

In the present invention, in order to compare the effect of SDF-1 using the dual ionic pH-sensitive copolymer, lysozyme was used as a model protein of SDF-1α Lysozyme has a similar size and a net charge as SDF-1α. 1 mg of Cy5.5-Lysozyme was applied to 10 mg of a dual ionic pH-sensitive copolymer to form micelles, and the tail vein was administered 3 hours after the preparation of the ischemic stroke rat model. After 3 hours and 21 hours, four mice each were euthanized and then the brain was removed, and cy5.5 fluorescence was detected by photographing near infrared fluorescence images. In the above experiment, as a control group, the group that induces only a stroke and the group that only Cy5.5-Lysozyme was administered were used. The results are shown in FIG. 10 and FIG. 11.

FIGS. 10 and 11 are diagrams confirming whether lysozyme encapsulated in a dual ionic pH-sensitive copolymer can be effectively delivered in an ischemic stroke animal model. FIG. 10 is a picture measuring the fluorescence intensity of Cy5.5 bound to lysozyme through near-infrared fluorescence imaging, and showing that the fluorescence intensity becomes stronger if it is almost yellow. FIG. 11 is a result of quantitative comparison of the fluorescence intensities of the ischemic ipsilateral hemisphere.

As shown in FIGS. 10 and 11, it was confirmed that lysozyme is accumulated as time passes in the ischemic ipsilateral hemisphere by a dual ionic pH-sensitive polymer. In contrast, lysozyme was not accumulated when only the normal hemisphere (contralateral hemisphere) and lysozyme were administered.

In addition, when these results are compared with Example 4 and FIGS. 4 and 5, 0.1 mg/ml in case of micelle in which SDF-1α is encapsulated, and 1 mg/ml in case of a micelle in which lysozyme is encapsulated were administered. Hence, it was confirmed that the brain lesion delivery effect of SDF-1α of a dual ionic pH-sensitive polymer of the present invention is significantly excellent as compared to lysozyme.

### Example 7: Drug Delivery Effect of Lysozyme of CMD (Carboxymethyle Dextran) in an Ischemic Stroke

CMD forms nanoparticles with hydrophobic nuclei and hydrophilic surfaces in a normal physiological environment and has a characteristic that nanoparticles collapse as the hydrophobic nuclei become hydrophilic in a hypoxic environment. This allows drug delivery to regions of cerebral ischemia with hypoxic conditions.

As a control group for comparison of the lysozyme delivery effect using a dual ionic pH-sensitive copolymer of the present invention, animal experiments were conducted to deliver lysozyme to the regions of cerebral ischemia using CMD.

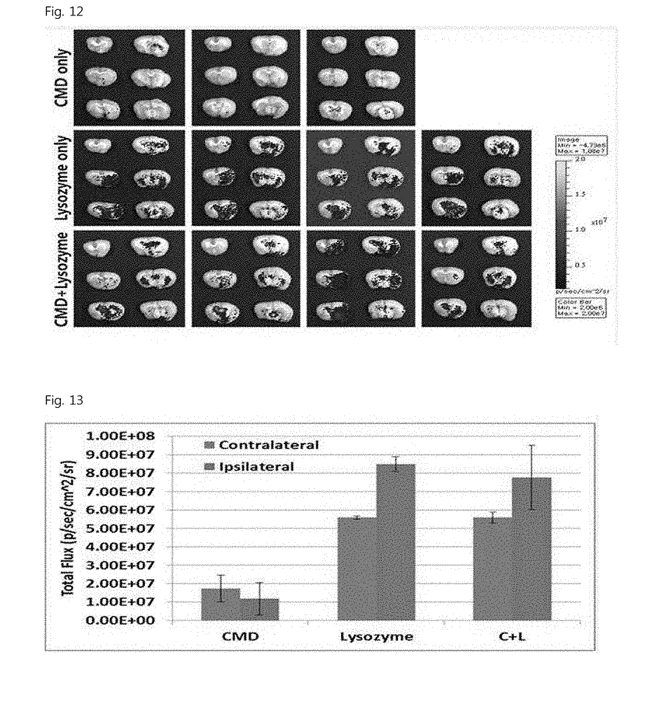

The results are shown in FIG. 12 and FIG. 13.

FIGS. 12 and 13 are the results of an experiment in which the delivery of lysozyme into stroke lesions was tried using Carboxymethyl Dextran (CMD), which is a substance used for drug delivery, such as a dual ionic pH-sensitive copolymer. FIG. 12 shows the results of near-infrared fluorescence imaging, and FIG. 13 shows the result of quantitative analysis of FIG. 12.

From the foregoing, it will be appreciated that various embodiments of the present invention have been described herein for purposes of illustration, and that various modifications may be made without departing from the scope and spirit of the present invention. Accordingly, the various embodiments disclosed herein are not intended to be limiting, with the true scope and spirit being indicated by the following claims.

1.-19. (canceled)

20. A method for treating an ischemic brain disease, the method comprising administering to a subject in need thereof a composition comprising an effective amount of stromal cell-derived factor-1 (SDF-1) and a dual ionic pH-sensitive copolymer, wherein the dual ionic pH-sensitive copolymer comprises: at a main chain, i) a repeating unit represented by the following Chemical Formula 1; and ii) a repeating unit represented by Chemical Formula 2 or Chemical Formula 3: 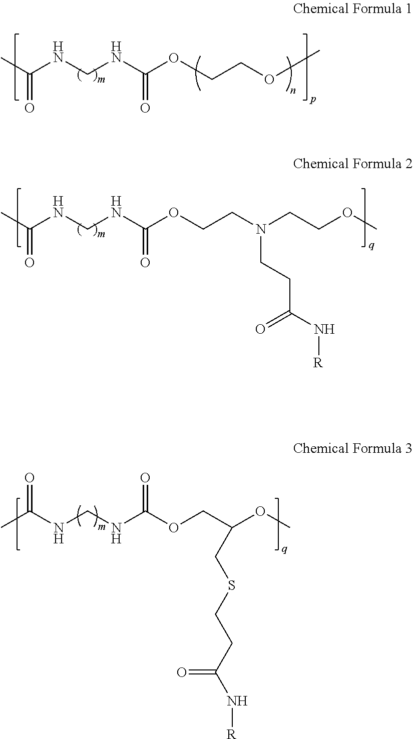
wherein m is an integer of from 4 to 6,
n is an integer ranging of 40 to 200,
p:q is from 1:1 to 1:10, and
each R is independently

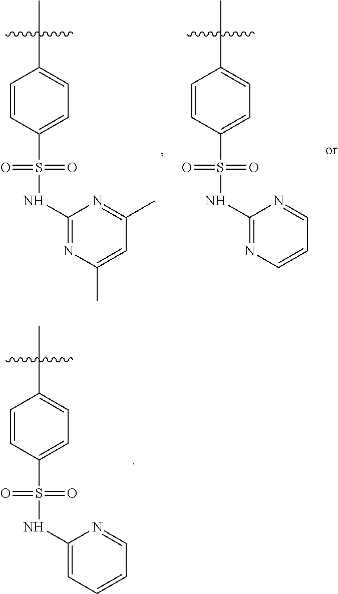

21. The method of claim 20, wherein a number average molecular weight of the dual ionic pH-sensitive copolymer ranges from 5,000 to 15,000.

22. The method of claim 20, wherein p:q ranges from 1:1 to 1:6.

23. The method of claim 20, wherein the dual ionic pH-sensitive copolymer is in the form of a self-assembled micelle.

24. The method of claim 23, wherein the SDF-1 is encapsulated in the micelle.

25. The method of claim 23, wherein the micelle is micellized at a pH range of from 4.5 to 8.5, and is demicellized at a pH below 4.5 or over 8.5.

26. The method of claim 23, wherein a diameter of the micelle ranges from 120 to 180 nm.

27. The method according to claim 20, wherein the SDF-1 is at least one selected from the group consisting of: SDF-1α, SDF-1β, SDF-1γ, SDF-1δ, SDF-1ε and SDF-1φ.

28. The method according to claim 20, wherein the ischemic brain disease is at least one selected from the group consisting of: palsy, stroke, cerebral hemorrhage, cerebral infarction, head injury, Alzheimer's disease, vascular dementia, Creutzfeldt-Jakob disease, coma, shock brain damage, and complications thereof.
